(19) United States
(12) Patent Application Publication
(10) Pub. No.: US 2013/0101528 A1
(43) Pub. Date: Apr. 25, 2013

(54) DENTIFRICE FORMULATION SUITABLE FOR PREGNANT OR NURSING WOMEN

(71) Applicant: Thanusha Govender, Umhlanga Rocks (ZA)

(72) Inventor: Thanusha Govender, Umhlanga Rocks (ZA)

(21) Appl. No.: 13/277,548

(22) Filed: Oct. 20, 2011

(51) Int. Cl. A61K 8/21; A61Q 11/00

(52) U.S. Cl. 424/52

(57) ABSTRACT

The invention relates to a dentifrice formulation which is particularly well suited for pregnant women, nursing women, or women trying to fall pregnant. The dentifrice formulation includes, as active ingredients: a source of fluoride ions, an abrasive agent, xylitol, and an anti-gingivitis agent; and as other ingredients: a surfactant, a humectant, a thickening agent, and an anti-inflammatory agent, thereby comprising therapeutic agents at least to control the incidence and severity of pregnancy gingivitis and tooth decay.

### FIELD OF INVENTION

This invention relates broadly to oral hygiene and more specifically to a dentifrice suitable for pregnant or nursing women.

### BACKGROUND OF INVENTION

The Inventor is aware that one of the major side effects of pregnancy is gingivitis and tooth decay. Gingivitis is a common form of gum disease known to develop in almost half of all pregnant women likely due to the change in hormonal levels. Pregnancy gingivitis may progress into a more serious periodontitis, if left effectively untreated, an advanced and irreversible form of gum disease.

Pregnant women with gum disease have a higher instance of preterm birth, a potentially serious pregnancy complication that may cause health concerns for their infant, typically due to a low birth weight. Babies who are born prematurely may risk a range of health conditions including cerebral palsy and problems with eyesight and hearing. Estimates suggest that about 18 out of every 100 premature births may be triggered by periodontal disease.

Bacteria that cause tooth decay are often passed from mother to infant who subsequently suffer from concerning health conditions.

The Inventor wishes to provide a dentifrice which may reduce the risk of pregnancy gingivitis and associated conditions. Accordingly, it is an object of the invention to provide a dentifrice which preventively addresses the oral care side effects developed during pregnancy due to hormonal changes within the body.

### SUMMARY OF INVENTION

In accordance with an embodiment, there is provided a dentifrice formulation which includes:
active ingredients including:
   a source of fluoride ions;
   an abrasive agent;
   xylitol; and
   an anti-gingivitis agent, and
other ingredients including:
   a surfactant;
   a humectant;
   a thickening agent; and
   an anti-inflammatory agent;
      thereby comprising therapeutic agents at least to control the incidence and severity of pregnancy gingivitis and tooth decay.

The dentifrice formulation may be in the form of a toothpaste formulation, although it may be adapted to take other forms (e.g. mouthwash, tooth powder, chewing gum, etc.). The dentifrice formulation is further described with reference to a toothpaste formulation, which is the preferred embodiment, though not necessarily the only embodiment.

Importantly, the toothpaste formulation is triclosan free. It may also essentially be alcohol free with the exception of xylitol which is a “tooth friendly” non-fermentable sugar alcohol that is safe for pregnant women. Regular use may reduce the probability of transmitting the Streptococcus mutans bacterium, which is responsible for tooth decay, from mother to child, during the first two years of life.

The toothpaste formulation contains a blend of ingredients that are safe in the concentration contained within this invention to be used during pregnancy and breastfeeding.

The toothpaste formulation may include optional ingredients selected from one or more of:
flavourants;
colourants;
preservatives; and/or
stain removal agents.

More particularly, the source of fluoride ions may be selected from sodium monofluorophosphate (MFP), Stannous Fluoride, Sodium Fluoride, or structures thereof. Preferably, the fluoride ion source is sodium monofluorophosphate (MFP). The sodium monofluorophosphate (MFP) may have a concentration in the formulation of between 400 ppm (parts per million) and 1,500 ppm, preferably 1,000 ppm.

The surfactant may be selected from one or more anionic, non-ionic, cationic, zwitter-ionic or amphoteric surfactants. Preferably, the surfactant source is sodium lauryl sulfate (SLS). The sodium lauryl sulfate (SLS) may have a concentration (m/m) in the formulation of between 1% and 4%, preferably 2%.

The abrasive agent may be selected from sodium bicarbonate (baking soda), calcium carbonate, silicas, dentin, di-calcium phosphates, calcium pyrophosphates, hydroxyapatites, trimetaphosphates, insoluble hexametaphosphates, or the like. Preferably, the abrasive agent is sodium bicarbonate. The sodium bicarbonate may have a concentration (m/m) in the formulation of between 10% and 50%, preferably 30%

The xylitol may have a concentration (m/m) in the formulation of between 3% and 36%, preferably 9.9%.

The humectant may be selected from glycerol, aqua, sorbitol, mixtures thereof, or the like. Preferably, the humectant is glycerin and aqua. The glycerine may have a concentration (m/m) in the formulation of between 5% and 30%, preferably 20%. The aqua may have a concentration (m/m) in the formulation of between 20% and 70%, preferably 33.4%.

The thickening agent may be selected from propylene glycol, carbomers, cellulose gum, xanthan gum, carrageenan, synthetic polymers, mixtures thereof, or the like. Preferably, the thickening agent includes propylene glycol with the added benefit of a polymer carbomer (934) which may create the desired rheology for the toothpaste formulation. The propylene glycol may have a concentration (m/m) in the formulation of between 0.01% and 0.2%, preferably 0.1%. The carbomer (934) may have a concentration (m/m, neutralised) in the formulation of between 0.1% and 1%, preferably 0.9%.

The anti-gingivitis agent may be carbamide peroxide or zinc citrate. Preferably, the anti-gingivitis agent is carbamide peroxide. The carbamide peroxide may have a concentration (m/m) in the formulation of between 1% and 10%, preferably 2.2%.

The anti-inflammatory agent may be allantoin, vitamin E, pro vitamin B5, mixtures thereof, or the like. Preferably, the anti-inflammatory agent is allantoin which may also serve to combat the potential side effects caused if SLS is the surfactant source. The allantoin may have a concentration (m/m) in the formulation of between 0.02% and 0.5%, preferably 0.1%.

The flavourant may be mint, such as peppermint oil. The peppermint oil may have a concentration (m/m) in the formulation of between 0.5% and 5%, preferably 1%.

The stain removal again may be sodium bicarbonate (baking soda), which has the additional functional benefit of being an anti-halitosis agent. Instead, or in addition, the stain removal agent may be urea peroxide.

### DETAILED DESCRIPTION OF EXAMPLE EMBODIMENT

A non-limiting example embodiment of a dentifrice formulation in the form of a toothpaste formulation will be further described. The toothpaste formulation includes:
active ingredients including:
   a source of fluoride ions;
   an abrasive agent;
   xylitol; and
   an anti-gingivitis agent, and
other ingredients including:
   a surfactant;
   a humectant;
   a thickening agent; and
   an anti-inflammatory agent;
      thereby comprising therapeutic agents at least to control the incidence and severity of pregnancy gingivitis and tooth decay.

| | | | Secondary | Concentration |
|---|---|---|---|---|
| Ingredient | Formula | Primary function | function | (m/m) |
| Sodium | Na₂FPO₃ | Fluoride ion source | Enamel re- | 1000 ppm F |
| monofluorophosphate | | to prevent tooth | mineralization | (0.76%) |
| | | decay | | |
| Sodium bicarbonate | NaHCO₃ | Anti-microbial | Whitening | 30% |
| (baking soda) | (9 microns) | abrasive | | |
| Xylitol | C₅H₁₂O₅ | Humectant & to | Sweetener | 9.9% |
| | | prevent tooth | | |
| | | decay | | |
| Carbamide peroxide | CH₆N₂O₃ | Anti-gingivitis agent | Whitening | 2.2% |
| Sodium lauryl sulfate | C₁₂H₂₉SO₄ | Surfactant/detergent | Anti-microbial | 2% |
| (SLS) | | | activity | |
| Glycerin | C₃H₈O₃ | Anti-septic & | Binder | 20% |
| | | Humectant | | |
| Propylene Glycol | C₃H₈O₂ | Binder | Humectant | 0.1% |
| Allantoin | C₄H₆N₄O₃ | Anti-inflammatory & | | 0.1% |
| | | anti-Swelling | | |
| Carbomer 934 | | Thickening agent/ | | 0.9% |
| | | polymer Binder | | (neutralized) |
| Aqua | H₂O | Humectant | | 33.4% |
| Peppermint oil | | Flavouring agent | | 1% |

The specific function of each constituent is described in more detail below:
Sodium monofluorophosphate (MFP): The role of the fluoride in the toothpaste is primarily to prevent tooth decay and make teeth stronger. Sodium MFP has been specially designed to protect tooth enamel from bacteria attack that causes dental caries (cavities).
Sodium bicarbonate (baking soda): It is the most multi-functional abrasive while being significantly non-abrasive and gentle making it ideal for pregnant women. Sodium bicarbonate exhibits both anti-plaque as well as anti-bacterial properties. It also neutralises acids within the oral cavity which assists in maintaining fresh breath. Baking soda is also effective in whitening or the reduction of staining of the natural dentition.
Sodium lauryl sulfate (SLS): It acts as a detergent in the toothpaste. It is responsible for creating foam which adds in the removal of food debris and it facilitates the dispersion of the active ingredients in the toothpaste. SLS displays anti-microbial properties.
Xylitol: It is a superior humectant thus enabling good toothpaste moisture and texture. Xylitol also acts as a safe sweetener and has virtually no after-taste. It also combats tooth decay and minimizes the transfer of streptococcus mutans bacterium from mother to child by 80%.
Peppermint oil (mentha piperita): It is a flavouring agent which is used to improve the taste of the toothpaste and to mask the after taste of SLS. The minty flavour is to enhance fresh breath.
Allantoin: It acts as a safe anti-swelling agent that soothes gums thus keeping the gums and oral cavity healthy. It also combats the potential side effect of obtaining canker sores due to the usage of SLS in the toothpaste.
Propylene glycol (E1520): It helps to bind non-binding ingredients. It connects and stabilises insoluble fluids (emulsifier) and it holds and dissolves active ingredients uniformly in the toothpaste while being able to retain moisture. It also has an added benefit of displaying antimicrobial properties. The low concentration used in the toothpaste is considered to be very safe.
Carbomer (934): It is used as a polymer binder which exhibits excellent rheological properties thus the formulated toothpaste will have very good stability, easy flow after stress and immediate restructuring upon stress cut-off. Carbomer 934 has an impressive shelf life and has a smooth and luxurious feeling.
Glycerin: It doubles up as both as a humectant and as a thickening agent. It also acts as an antiseptic.
Carbamide peroxide: The low concentration used in the toothpaste is considered to be pregnancy safe. It is effective in reducing gingival inflammation and supragingival plaque formation. Carbamide peroxide is an antiseptic agent that is effective in enamel remineralisation, reduction in tooth decay and as a whitening agent.
Aqua: It is used as a humectant.

The embodiment as exemplified thus provides a luxurious toothpaste designed for pregnant women that provides total teeth and gum protection during their pregnancy and into early stages of motherhood. It can also be included into the mothers to be pre-natal care regime.

More specifically, the example embodiment provides a multi-functional toothpaste that preventively addresses the oral care side effects developed during pregnancy due to hormonal changes within the body. It is designed for pregnant women and hence all the ingredients are in concentrations that are safe for oral use during pregnancy and breastfeeding

The example embodiment provides the following oral care benefits:
holistic gum protection (it provides anti-plaque, anti-bacterial and anti-swelling protection);
cavity protection;
non-abrasive stain removal; and
it reduces halitosis.

1. A dentifrice formulation which includes:
active ingredients including:
a source of fluoride ions;
an abrasive agent;
xylitol; and
an anti-gingivitis agent, and
other ingredients including:
a surfactant;
a humectant;
a thickening agent; and
an anti-inflammatory agent;
thereby comprising therapeutic agents at least to control the incidence and severity of pregnancy gingivitis and tooth decay.

2. A dentifrice formulation as claimed in claim 1, which further includes ingredients selected from:
flavourants;
colourants;
preservatives; and/or
stain removal agents.

3. A dentifrice formulation as claimed in claim 1, in which the source of fluoride ions is selected from sodium monofluorophosphate (MFP), Stannous Fluoride or Sodium Fluoride.

4. A dentifrice formulation as claimed in claim 3, in which the source of fluoride ions is sodium monofluorophosphate (MFP) having a concentration in the formulation of between 400 ppm (parts per million) and 1,500 ppm.

5. A dentifrice formulation as claimed in claim 4, in which the sodium monofluorophosphate (MFP) has a concentration in the formulation of 1,000 ppm.

6. A dentifrice formulation as claimed in claim 1, in which the surfactant is selected from at least one anionic, non-ionic, cationic, zwitter-ionic or amphoteric surfactant.

7. A dentifrice formulation as claimed in claim 6, in which the surfactant source is sodium lauryl sulfate (SLS) having a concentration (m/m) in the formulation of between 1% and 4%.

8. A dentifrice formulation as claimed in claim 7, in which the lauryl sulfate (SLS) has a concentration (m/m) in the formulation of 2%.

9. A dentifrice formulation as claimed in claim 1, in which the abrasive sources is selected from at least one of sodium bicarbonate (baking soda), calcium carbonate, silicas, dentin, di-calcium phosphates, calcium pyrophosphates, hydroxyapatites, trimetaphosphates, or insoluble hexametaphosphates.

10. A dentifrice formulation as claimed in claim 9, in which the abrasive source is sodium bicarbonate having a concentration (m/m) in the formulation of between 10% and 50%.

11. A dentifrice formulation as claimed in claim 10, in which the sodium bicarbonate has concentration (m/m) in the formulation of 30%.

12. A dentifrice formulation as claimed in claim 1, in which the xylitol has a concentration (m/m) in the formulation of between 3% and 36%.

13. A dentifrice formulation as claimed in claim 12, in which the xylitol has a concentration (m/m) in the formulation of 9.9%.

14. A dentifrice formulation as claimed in claim 1, in which the humectant is glycerin and aqua.

15. A dentifrice formulation as claimed in claim 14, in which the glycerine has a concentration (m/m) in the formulation of between 5% and 30% and the aqua has a concentration (m/m) in the formulation of between 20% and 70%.

16. A dentifrice formulation as claimed in claim 15, in which the glycerine has a concentration (m/m) in the formulation of 20% and the aqua has a concentration (m/m) in the formulation of 33.4%.

17. A dentifrice formulation as claimed in claim 1, in which the thickening agent is selected from one of propylene glycol, carbomers, cellulose gum, xanthan gum, carrageenan, or a synthetic polymer.

18. A dentifrice formulation as claimed in claim 17, in which the thickening agent is propylene glycol and a polymer carbomer (934).

19. A dentifrice formulation as claimed in claim 18, in which the propylene glycol has a concentration (m/m) in the formulation of between 0.01% and 0.2% and the carbomer (934) has a concentration (m/m, neutralised) in the formulation of between 0.1% and 1%.

20. A dentifrice formulation as claimed in claim 19, in which the propylene glycol has a concentration (m/m) in the formulation of 0.1% and the carbomer (934) has a concentration (m/m, neutralised) in the formulation of 0.9%.

21. A dentifrice formulation as claimed in claim 1, in which the anti-gingivitis agent is selected from carbamide peroxide or zinc citrate.

22. A dentifrice formulation as claimed in claim 21, in which the anti-gingivitis agent is carbamide peroxide having a concentration (m/m) in the formulation of between 1% and 10%.

23. A dentifrice formulation as claimed in claim 22, in which the carbamide peroxide has a concentration (m/m) in the formulation of 2.2%.

24. A dentifrice formulation as claimed in claim 1, in which the anti-inflammatory agent is selected from allantoin, vitamin E, pro vitamin B5, or mixtures thereof.

25. A dentifrice formulation as claimed in claim 24, in which the anti-inflammatory agent is be allantoin having a concentration (m/m) in the formulation of between 0.02% and 0.5%.

26. A dentifrice formulation as claimed in claim 25, in which the allantoin has a concentration (m/m) in the formulation of 0.1%.

27. A dentifrice formulation as claimed in claim 1, in which the flavourant is peppermint oil having a concentration (m/m) in the formulation of between 0.5% and 5%.

28. A dentifrice formulation as claimed in claim 2, in which includes a stain removal agent selected from sodium bicarbonate (baking soda) and/or urea peroxide.

29. A dentifrice formulation as claimed in claim 1, which is in the form of a toothpaste formulation.

30. A dentifrice formulation as claimed in claim 29, in which the toothpaste formulation is particularly well suited for pregnant women, nursing women, or women trying to fall pregnant.
